(19) United States
(12) Patent Application Publication
(10) Pub. No.: US 2015/0157475 A1
(43) Pub. Date: Jun. 11, 2015

(54) DEFLECTOR FOR INCREASED WALL SHEAR STRESS ADJACENT AN ARTERIOVENOUS FISTULA

(71) Applicant: Abbott Cardiovascular Systems Inc., Santa Clara, CA (US)

(72) Inventors: Paul Consigny, San Jose, CA (US); Jesus Managa, Redwood City, CA (US)

(73) Assignee: Abbott Cardiovascular Systems Inc., Santa Clara, CA (US)

(21) Appl. No.: 14/099,206

(22) Filed: Dec. 6, 2013

(51) Int. Cl. A61F 2/82; A61F 2/95

(52) U.S. Cl. CPC A61F 2/82; A61F 2/95

(57) ABSTRACT

A medical device includes an elongate body having a distal and proximal end, a pair of anchors at the distal end and spacers extending along its length. The body is delivered to an AV fistula using a catheter connected to the proximal end by a tether. The body when deployed at the fistula is suspended within the flow stream and spaced from walls of a blood vessel. The body deflects blood flow to cause an increase in vascular wall shear stress, which has been found to induce a positive vascular remodeling.

### FIELD OF THE INVENTION

The present invention relates to implantable medical devices associated with the creation of, and/or the maturation of an arteriovenous (AV) fistula or bypass graft access structure for hemodialysis.

### BACKGROUND OF THE INVENTION

AV Fistula (a connection between an artery and a vein) are a desired access structure for the dialysis of kidney failure patients. FIG. 1 illustrates a matured portion of the vein near the artery, which acts as a re-usable cannula access site proximal the AV fistula.

About 42% of surgically created AV Fistula fail to mature; that is, the portion of the vein proximal the fistula fails to adapt physiologically to accommodate the higher arterial pressure. When this venous portion (or side of the AV fistula) matures, it becomes usable as a cannula access site for dialysis (FIG. 1). Maturation can take about 6 weeks from forming the fistula. Failure to mature and/or act as a good dialysis access site is most commonly the result of poor blood flow (low blood pressure/low blood flow rates) in the venous portion of the fistula. About 74% of these failures are salvaged by some form of intervention, followed by maturation of the venous side in another 6-8 weeks. The remaining about 11% of the cases are regarded as failures, which necessitates creating an AV Fistula at another site. The most common site of initial AV Fistula creation is the wrist. If a new AV Fistula is required, a new site proximal of the previous/failed site is chosen. Typically, there are 3 potential sites per arm.

Patients without a mature AV Fistula require some other, less desirable form of dialysis access for the standard 3 times a week dialysis regimen until a mature fistula is available. Additionally, about a third of mature fistula fail in a year. The health of kidney failure patients without a functioning mature AV Fistula deteriorates at a more rapid rate than those with one. Deteriorating health makes the subsequent creation of a functioning mature AV Fistula less probable, necessitating a significant number of interventions or access procedures resulting in poorer survival rates. Thus, a significant number of interventions and procedures may be avoided or significantly delayed, significant cost savings realized and the survival rate of dialysis patients significantly improved by decreasing the failure to mature rate of newly created AV fistula and by reducing the rate at which mature fistula fail.

AV fistula failure to mature is often tied to the development of a neointimal hyperplasia that occurs in areas adjacent to the arteriovenous anastamosis. The neointimal hyperplasia develops secondarily to turbulent blood flow at the anastamosis. A major contributor to this turbulence is believed to lie in the combination of retrograde and antegrade flow at the anastomosis—arterial flow coming towards the fistula not only from the upstream artery but also the downstream artery via collateral flow. See Bettinger, C J et al. Three-dimensional microfluidic tissue-engineering scaffolds using a flexible biodegradable polymer. Adv Mater 18: 165-169, 2005; and Guan, J et al. Preparation and characterization of highly porous, biodegradable polyurethane scaffolds for soft tissue applications. Biomaterials 26: 3961-71, 2005.

Previous studies document that increases in vascular wall shear stress induce vascular enlargement (positive vascular remodeling). This remodeling is thought to be an endothelium-dependent process where increased flow or shear stress is transduced by the endothelium into signals such as the production of metalloproteinases that breakdown the extracellular matrix and permit vessel expansion. See Carlier, S G et al. Augmentation of wall stress inhibits neointimal hyperplasia after stent implantation: inhibition through reduction of inflammation? American Heart Assoc., Circulation 107: 2741-46, 2003 (May 2003).

A flow augmentation device called an Anti-Restenotic Diffuser or ARED was previously proposed for increasing wall shear stress. Murphy, Eoin A. and Boyle, Fergal J., Reducing in-stent restenosis through a novel stent flow field augmentation. Vol. 4, No. 3 Cardiovascular Engineering and Technology, pp. 353-373 (December 2012) (Murphy). Similar devices are also described in U.S. Pat. No. 6,641,605 ('605 patent) and EP0989830. Referring to FIGS. 1-2 of the '605 patent, there is demonstrated the effects of placing a deflector 1 in the bloodstream: a greater radial gradient of velocity is created. This higher gradient increases the shear stress at the blood-wall of the artery. Also discussed are ratios of deflector to artery radii for purposes of increasing the shear near an artery wall. See Col. 2, line 62 through col. 5, line 3. A proposed deflector having coiled springs is shown in FIGS. 4-5 of the '605 patent.

There is a need to provide a deflector for increased wall shear stress that can be effectively placed adjacent an AV fistula to decrease the failure to mature rate. While the known deflectors intended for reducing restenosis are helpful in achieving this goal, they do not fully and/or adequately address these needs.

### SUMMARY OF THE INVENTION

This invention is designed to be implanted in an artery or vein for the purposes of stimulating flow-induced positive remodeling. Such remodeling would improve the function of bypass grafts and arteriovenous fistulae.

This invention is a device that is implanted within the lumen of a blood vessel. The device can be either a removable device or a permanent implant. The device occupies the central part of the lumen of the vessel thereby diverting blood flow and increasing blood velocity at the vessel wall. The resulting increase in velocity results in an increase in shear stress that promotes vessel enlargement.

According to one aspect a medical device includes spacers for spacing a flow deflecting body away from walls of a vessel, and anchors for maintaining the body's position in the vessel. The anchors and spacers may be made from a shape memory material such as nitinol wire.

According to another aspect a medical device includes a flow deflector delivery system including a catheter and flow deflector configured for being implanted near an anastomosis.

In accordance with the foregoing, there is a flow deflector, medical device, method of use, method for making, or method for assembly of a medical device comprising such a flow deflector, and a delivery system including such a flow deflector having one or more, or any combination of the following things (1)-(18):
(1) A flow deflector according to any of the embodiments in Table 1 and/or Table 2.
(2) A catheter and flow deflector.
(3) A body having spacers configured to extend from a surface of the body
(4) A body having a diameter of about 1 to 3 mm.
(5) A body having a length of 2 to 10 mm.
(6) A flow deflector having 3, 6 or 9 spacers as shown and described in any of the drawings or examples disclosed herein.
(7) Spacers for a flow deflector arranged as longitudinally aligned, or offset spacers, or combination of the two.
(8) Anchors of the flow deflector holding the flow deflector within the lumen and resisting movement of the flow deflector due to drag forces caused when fluid is being diverted by the body of the flow deflector. The anchors being configured in two positions: a collapsed or stowed configuration and a deployed, expanded or extended configuration. When the stowed configuration the anchors are contained within a sheath. When the expanded configuration the anchors extend outwardly to hold the flow deflector at or near an anastomosis, wherein the anastomosis is either formed in part by a vascular access graft or is an AV fistula.
(9) Longitudinally aligned spacers. At least 2 or 3 spacers are longitudinally aligned.
(10) Offset spacers. At least 2 or 3 spacers are longitudinally offset form each other. The spacers may be arranged in a helical pattern over the length of the body, or a plurality of sets of three or more spacers, spacers in each set being longitudinally aligned.
(11) A flow deflector including stabilizing supports that maintain the body away from walls of the vessel. The supports are biased to deploy outward from a surface of the body to a maximum height h-max, wherein h-max is the greatest distance outward from the outer surface of a body, e.g., “h” is defined in terms of a preferred embodiment in FIGS. 6A-6B. H-max may correspond to the maximum height when no radial constraint is placed upon the flow deflector. When the flow deflector is in a sheath the support has a height h1 that may be about zero; when placed within a vessel the support extends outward until it reaches the wall of a vessel, whereupon it achieves a second height h2. As the vein continues to mature the vein lumen wall increases in size and the height of the support increases until it arrives at h-max.
(12) A flow deflector having a minimum of three circumferentially spaced supports spring-biased to radially extend outward from a surface of the body so that a generally circular radial outward force is imposed on vessel walls. The at least three spacers providing a generally circular radial outward force may together extend over about the entire length of the body, ½ of the body, or ⅓ of the body.
(13) A flow deflector including an elongate body having a distal end and a proximal end; an anchor configured to extend distally of the distal end and radially outward from the body; and a plurality of spacers configured to extend from a surface of the body.
(14) The aspects of disclosure as set forth in (13), (15), (17) or (18), in combination with one of, more than one of, or any combination in any order of the following list of things: wherein the body is made from a polymer, metal or metal alloy; wherein the body includes a lumen for receiving a guide wire; wherein the body is cylindrical; wherein the body has a length to diameter ratio of about 20:1, 30:1, 40:1 or between about 20:1 to 30:1 or between about 20:1 to 40:1; wherein the anchors are configured between a deployed and stowed form, wherein when in the stowed form the anchors have a maximum radial extent less than or equal to the diameter of the body; wherein the anchors are made from a shape memory material; wherein the anchors comprise a pair of wings or petals; wherein the spacers are made from a shape memory material; wherein at least three spacers are configured to extend from the body surface; wherein a spacer includes a first end, second end and a center portion configured to extend from the body surface, wherein the first and second ends are disposed within the body and the center portion is exterior of the body surface; wherein the first end is fixed to the body and the second end is disconnected from the body; wherein a spacer is a wire that has first and second ends received in a lumen of the body, and a center portion configured to extend outward from the body surface; wherein the center portion of the wire extends out from a first hole and into a second hole, the first and second holes each being formed in the body surface and in communication with the body lumen; wherein the body has a length (L) and the first and second holes are separated by a length equal to about ½*L, ⅓*L, ¼*L, or between about ⅕*L and ½*L; wherein the body has a diameter D and each one of, or any combination of the spacers are configured for radially extending from the body surface by up to about ½*D, 2*D, 3*D, 4*D, and *5D; wherein the flow deflector has three or six spacers; and/or wherein a spacer is longitudinally and/or radially aligned with at least one other spacer or offset and/or radially spaced from at least one other spacer.
(15) A flow deflector delivery system including a flow deflector, the flow deflector including an elongate body having a distal end and proximal end, and a connector disposed at the proximal end; and a catheter having a distal end, including a tether engaged with the connector, and a sheath encapsulating the flow deflector.
(16) The aspects of disclosure as set forth in (13), (15), (17) or (18), in combination with one of, more than one of, or any combination in any order of the following list of things: wherein the connector comprises a loop, a hook and/or an eyelet; wherein the body includes anchors disposed at the body distal end, the anchors being contained within the sheath and exterior of the body; further including a guide wire lumen in the body, wherein a guide wire may be extended from a catheter lumen through the body guide wire lumen; the catheter further including at a distal end a pusher, wherein the connector is received within the pusher; wherein the connector is a looped member disposed at the proximal end thereof; and/or wherein the tether is retractable and releasable from the connector.
(17) A kit including a catheter and a flow deflector. The kit may also include a retrieval snare or device.
(18) A method of treatment for a dialysis access structure, comprising: intravenously delivering a body at the dialysis access structure; positioning the body at a vascular anastomosis such that the body is spaced from walls of a blood vessel near the anastomosis; and wherein the dialysis access structure is one of an AV fistula or vascular bypass graft.

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### BRIEF DESCRIPTION OF THE DRAWINGS

FIG. 1 is a side-view of the arm of a patient receiving dialysis. A fistula is shown.

FIG. 2 shows an exploded view of a delivery system and flow deflector according to the disclosure. The flow deflector includes a body, anchors and spacers.

FIG. 3 shows a similar disclosure of the flow deflector of FIG. 2 when implanted at an arteriovenous (AV) fistula. Alternatively, the device may be placed at an anastomosis formed by a bypass graft, such as the bypass grafts described in US 2007/0142897.

FIG. 4 shows a similar disclosure of the flow deflector of FIG. 2 with flow deflector configured for intraluminal delivery to a fistula. There is the flow deflector including the body, anchors and spacers encapsulated within a sheath of the catheter. The device is steered over a guide wire towards the fistula.

FIG. 5A shows the assembly for spacers of the flow deflector according to FIG. 2, 3 or 4 when the flow deflector is encapsulated within the delivery sheath of the catheter. The spacer is stowed partially or substantially within a storage lumen of the body of the flow deflector.

FIG. 5B shows the assembly of the spacer of FIG. 5A with the sheath removed and the spacer fully or partially deployed from a storage lumen.

FIGS. 6A-6B show two embodiments of shape-memory wire for forming spacers.

FIG. 7A shows an assembly of spacers for a flow deflector according to the disclosure where spacers are longitudinal offset from each other, such as for the flow deflector of FIG. 2.

FIG. 7B shows an assembly of spacers for a flow deflector according to the disclosure where the spacers are longitudinal aligned from each other, such as for the flow deflector of FIG. 3.

FIG. 7C is a cross-sectional frontal view of the flow deflector according to the disclosure. There is shown six spacers spaced 60 degrees apart. The spacers maintain the body within the center of the vessel and spaced away from blood vessel walls.

FIG. 8 shows a proximal end of the body of the flow deflector.

FIG. 8A shows a connector including an eyelet and hook for attachment at the proximal end of the body.

FIG. 9 shows a distal end of the body of the flow deflector with anchors attached.

FIGS. 9A-9B illustrates a wire forming the anchors in for the flow deflector, arranged in a stowed and deployed state.

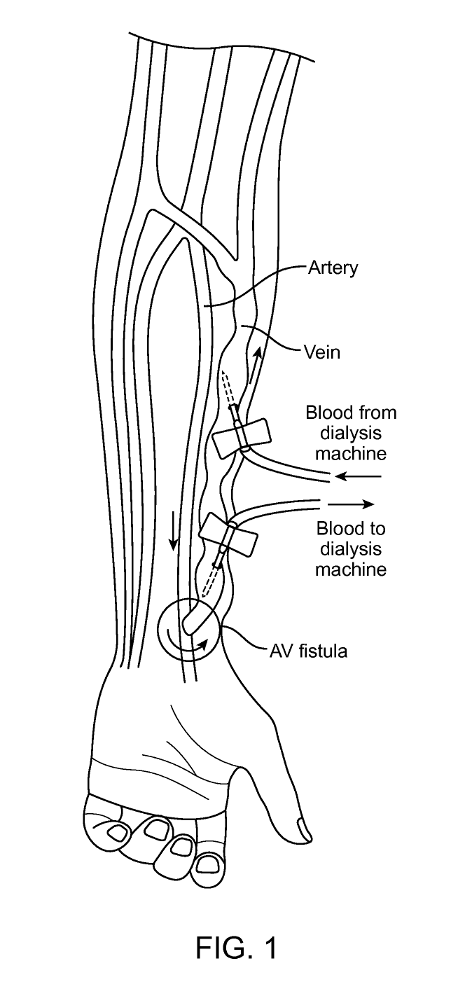

### DETAILED DESCRIPTION OF EMBODIMENTS

For purposes of this disclosure, the following terms and definitions apply:

When referring to a vein or artery prior to making a fistula, a “proximal end” refers to an end closest to the torso of the body, whereas a “distal end” refers to the end furthest from the torso of the body. In contrast, after the fistula is made, when referring to a medical device's intended location relative to a fistula or anastomosis, the terms “proximal” and “distal” are instead made with respect to the relative location of the fistula or anastomosis. Thus, for example, the end of a scaffold closest to the fistula will be called the “proximal” end and the end furthest from the fistula the “distal” end. Thus, generally speaking, prior to making the fistula the former terminology is used. And after the fistula is made “proximal” and “distal” always refers to a location relative to the fistula.

The term “about” means 20%, 15%, 10%, 5%, 4%, 3%, 2%, 1.5%, 1%, between 1-2%, 1-3%, 1-5%, or 0.5%-5% less or more than, less than, or more than a stated value, a range or each endpoint of a stated range, or a one-sigma, two-sigma, three-sigma variation from a stated mean or expected value (Gaussian distribution). It is understood that any numerical value, range, or either range endpoint (including, e.g., “about none”, “about all”, etc.) preceded by the word “about” in this disclosure also describes or discloses the same numerical value, range, or either range endpoint not preceded by the word “about”.

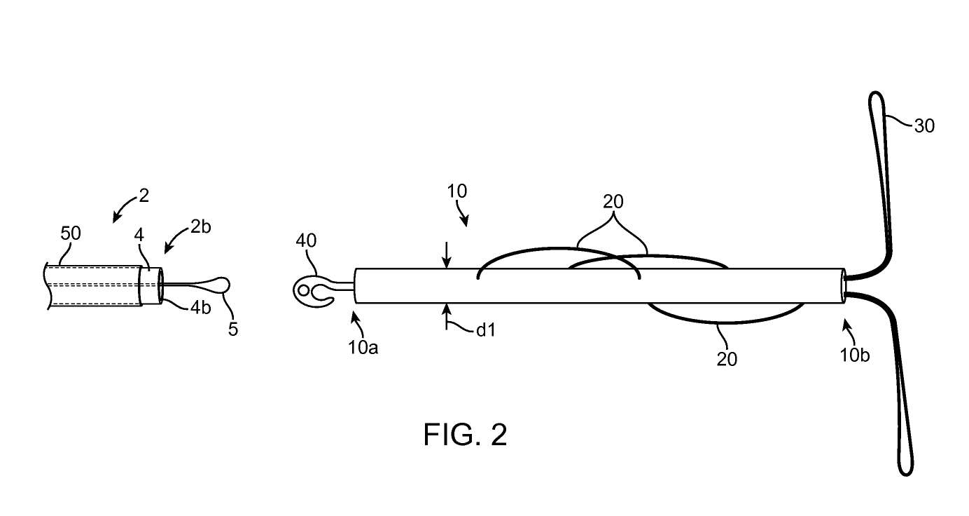

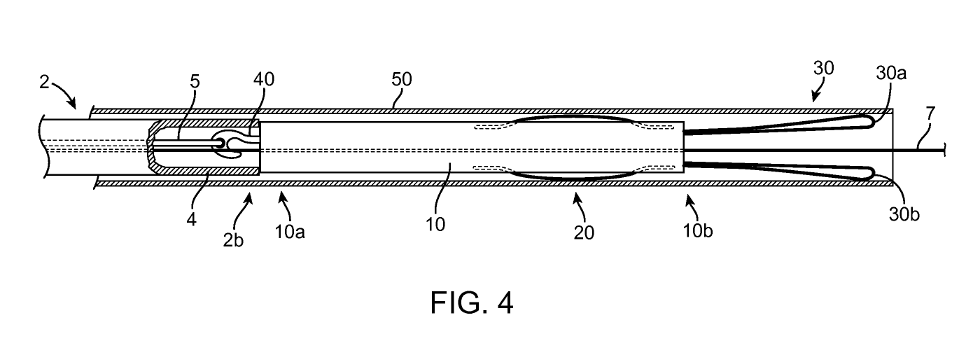

FIG. 2 shows a flow deflector system according to one aspect of the disclosure. The system includes a flow deflector and catheter 2 configured to deliver and place the flow deflector at an arteriovenous (AV) fistula intravenously. In FIG. 2 the flow deflector and catheter 2 are shown in an exploded view for illustrative purposes. FIG. 4 shows the flow deflector and catheter when configured for intraluminal delivery to the fistula site.

The flow deflector includes a tubular body 10 that may be an extruded or molded polymer, or metal tube. The body 10 is cylindrical with a length to outer diameter ratio of about 20:1, 30:1, 40:1 or between about 20:1 to 30:1 or between about 40:1 to 40:1. Thus, according to the disclosure the body 10 is elongate with a length up to 40 times the diameter. The body 10 has a proximal end 10a, which includes a connector 40 used to connect the body 10 to the catheter 2 distal end 2b, which has a mating connector, e.g., a tether 5. The connector 40 may also be used to retrieve the flow deflector from the fistula after it has served its purpose. The connector 40 may be shaped as needed so that it can be engaged by a variety of commercially available vascular snares. Examples of snares that may be used are those described in U.S. Pat. No. 8,177,790, US2011/0264106, and US2012/0289945.

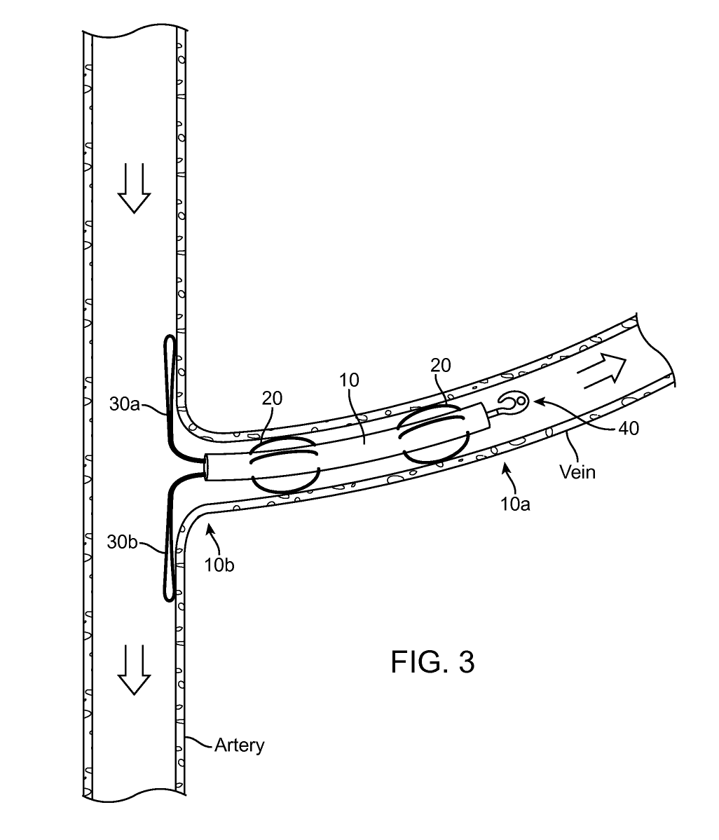

The flow deflector also includes anchors 30 disposed at a distal end 10b of the body 10. The anchors 30 help to hold the flow deflector in place at the fistula, as shown in FIG. 3. In the illustrated embodiments the anchors include a pair of wings or petals 30 that are made from a shape memory material such as nitinol wire. The petals 30 self-expand to the position shown in FIG. 2 when the body distal end 10b is freed from a delivery sheath 50 portion of the catheter 2 (FIG. 4 shows anchors 30 when stowed in the delivery sheath 50).

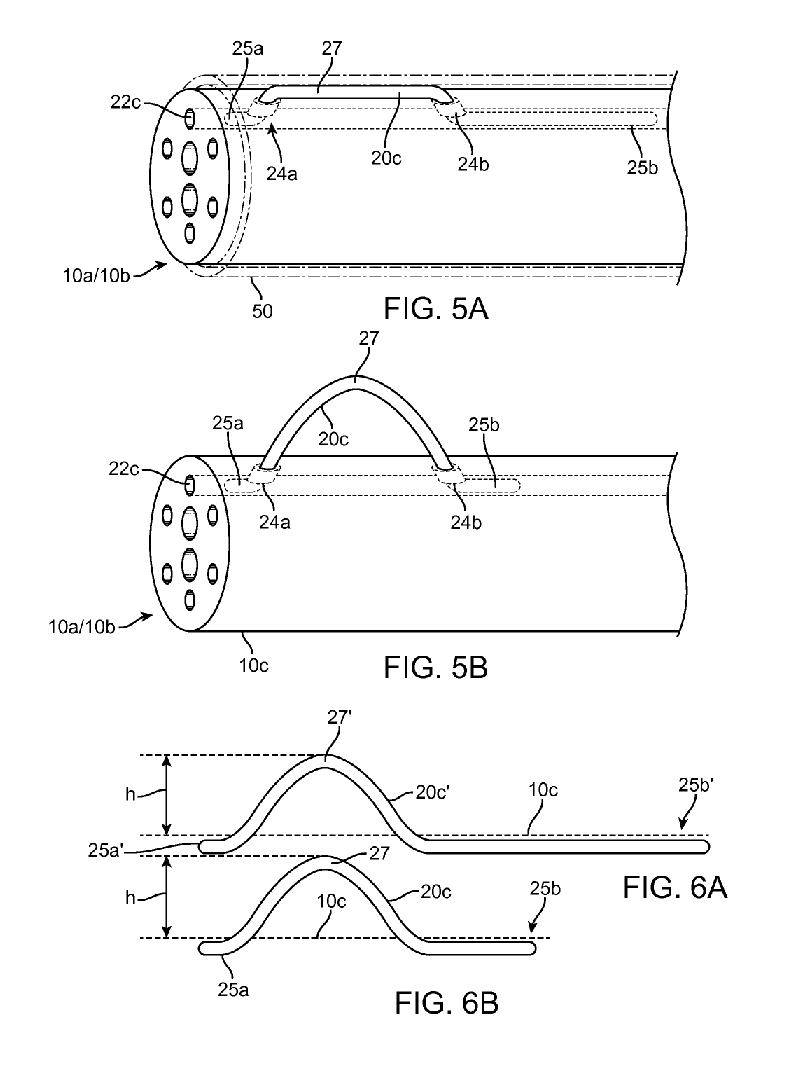

The flow deflector further includes spacers 20 (three shown in FIG. 2) configured to maintain the body 10 in the center of the flow stream when they are in a deployed configuration, such as shown in FIG. 3. In this way the body deflects the blood flow towards the walls, thereby increasing wall shear stress. The spacers 20 may be made from shape-memory nitinol wire. The spacers 20 are stowed within lumen of the body 10 when the body 10 is within the delivery sheath 50, as shown in FIGS. 4 and 5A. When the sheath 50 is removed the 20 spacers extend outwards to take the shape shown in FIGS. 2 and 3.

During placement at the fistula, the tether 5 connection to the hook 40 allows the body 10 to be drawn back or pulled towards the catheter 2 proximal end (not shown) to adjust placement at the fistula (as needed). Forward placement, i.e., displacement of the body 10 distal of the distal end 2a of the catheter 2, is accomplished by a distal end 2b having a surface that abuts the end 10a. This distal end 2b is a pusher 4 configured to push the body 10 forward or distally, either for fine adjustments or to assist with removing body 10 from the sheath 50, which encapsulates the body 10 during delivery to the AV fistula site, as shown in FIG. 4. After the body 10 is positioned properly at the fistula, the catheter 2 may be maneuvered including axial movement of the tether 5 to release the tether 5 from the connector 40. After the tether 5 is released from the connector 40 the catheter 2 may be withdrawn and removed from the patient.

Referring to FIG. 3 there is shown the flow deflector implanted at the fistula. The anchors 30 hold the body 10 in place, while the spacers 20 keep the body 10 in the center of the flow stream. The anchors 30a, 30b are pre-shaped to extend outward naturally (when free from the sheath 50) to hold the body 10 at the anastomosis as shown. The wires forming the anchors 30a, 30b have a sufficiently high flexural stiffness to resist being pulled into the vein by drag forces on the body 10. The connector 40 is exposed at the proximal end 2a so that the flow deflector may be removed from the patient using a snare.

The outer diameter of the body 10 may be chosen as ½, ⅓ or ¼ of the average inner diameter of a vein lumen before or after maturation of the vein lumen. For example, the body may have a diameter of about 1, 2, or 3, or about 1 to 3 mm and the length of the body may be about 3 to 10 mm. The number of spacers 20 may vary. FIG. 2 shows a body with three spacers 20, whereas FIG. 3 shows a body 10 with six spacers 20—there are three spacers 20 longitudinally aligned and nearest the proximal end 10a and three longitudinally aligned spacers 20 nearest the distal end 10b in this embodiment of a deflector. Alternatively the six spacers may be evenly spaced (longitudinally) over the length of body 10 to form a helical pattern of spacers 20.

In some embodiments the number of spacers can be 9, or up to 9 spacers. In the case of a body that is relatively long, i.e., about 10 mm, it is preferred to have as much as 9 spacers for maintaining the body within the center of the flow stream. For example, the flow deflector may be arranged to have nine spacers arranged in a helical pattern so that each spacer is longitudinally spaced from an adjacent spacer by an equal amount; and/or the spacers are arranged to describe a helical pattern that encompasses one, or more than one revolution; that is, 360 Degrees about a circumference of the body; and or some of the spacers can be longitudinally aligned while others are longitudinally spaced such as described in the drawings. For example, spacers at one of the proximal or distal end can be longitudinally aligned while the other of the proximal and distal ends can be longitudinally offset, or describe a full or partial helical shape about the body circumference; and/or the spacers can be arranged in three sets of three spacers, each of which are longitudinally and each set is longitudinally offset with the other, e.g., the flow deflector of FIG. 3 with an additional set of the spacers in the middle of the body.

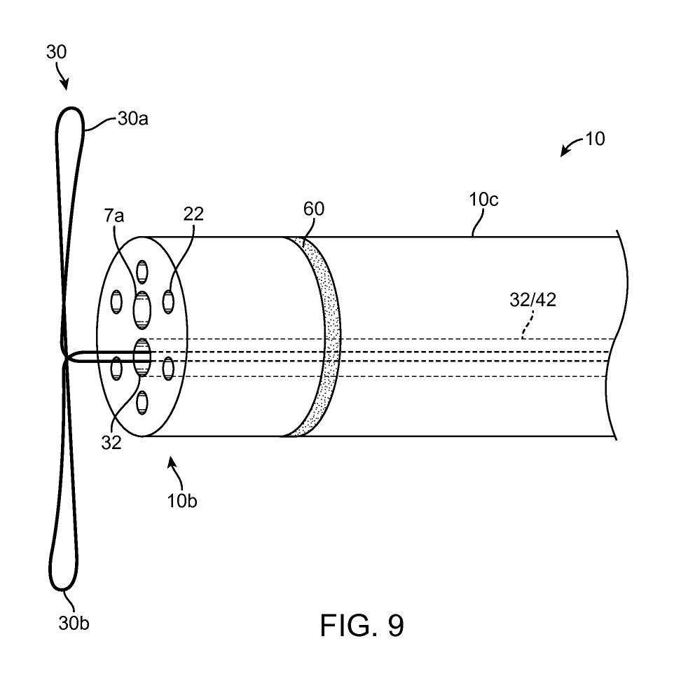

The number and position of the spacers 20 can vary provided the spacers 20 are able to stably hold the body 10 within approximately the center of the flow stream during the maturation period (when the vein inner diameter substantially increases in size about 2-5 times). To this end, in some embodiments there is at least three spacer elements circumferentially spaced (e.g., helical) so that the body 10 can be stably maintained within the middle of the flow stream. In FIG. 2 there are three spacers that are circumferentially spaced 120 degrees apart. In some embodiments spacers 20 are placed closer the distal end 10b than the proximal end 10a as the distal end 10a, being about at the fistula, is likely to experience more turbulent or circular flow than the proximal end. Alternative embodiments of spacers 20 are shown in FIGS. 5, 5A and 8, and TABLE 1 infra.

Referring to FIG. 4 there is shown in partial cross-sectional view the deflector delivery system as it would appear when the deflector is delivered intravenously to the fistula. There is shown the sheath 50 encapsulating the anchors 30, body 10 and spacers 20, thereby reducing the profile of the deflector for passage through the vessel lumen. The sheath 50 has a connection at a proximal end (not shown) to allow the operator to withdraw the sheath 50 when the distal end 2b has arrived at the fistula and the deflector is in position for deployment. As shown the anchors 30 and spacers 20 are in a stowed position. The spacers 20 of FIG. 4 are placed only closest to the distal end 10b, as opposed to the FIG. 3 spacers 20, which are located both at the distal and proximal ends. Also, as mentioned earlier, in illustrated embodiments the spacer 20 retracts within lumens of the body 10, as explained in more detail below in connection with FIGS. 5A-5B. At the proximal end 10a of the body 10 the connector 40 is engaged with the tether 5. The pusher 4 has a lumen sized to receive the connector 40 which is engaged by the tether 5. With the tether 5 connected to the connector 40 and taught, and sheath 50 covering the body 10, the body 10 is stably held at the catheter 2 distal end may be delivered.

A preferred pathway for the flow deflector would be as follows: femoral vein, iliac vein, inferior vena cave, superior vena cava, subclavian vein, brachial vein and then cephalic vein. This same pathway could also be used for delivery of a retrieval device for removing the flow deflector from the patient. The removal of the flow deflector (when engaged with the retrieval device) would then proceed in the reverse order. An alternative route would be insertion in the brachiocephalic vein of the arm and then advance the catheter towards the wrist and site of the fistula.

With reference to FIGS. 3 and 4, the method of deployment at the fistula may proceed as follows. As alluded to earlier, the pusher 4, tether 5, and sheath 50 may be used to deploy and place the body 10 as desired. A guide wire 7 is first placed in the vessel and the catheter 2 passed over the guide wire (the body 10 preferably also includes a guide wire lumen, in addition to the catheter 2). Via passage over the guide wire 7 the distal end of the sheath 50 is placed at the fistula or slightly downstream or upstream of it. Preferably a radiopaque marker band is provided on the sheath 50 and/or body 10 distal ends for visual tracking of the location of the catheter distal end 2b and/or body 10 relative to the fistula.

The sheath 50 is withdrawn while the pusher 4 is held in place or pushed slightly distally to push the anchors 30 outward and deploy. As mentioned earlier, the anchors 30a, 30b are pre-shaped so that when freed from the sheath 50 they naturally unfold to rest against the nearby upstream and downstream walls of the artery, as shown in FIG. 3. Once the wings 30a, 30b are in place, the sheath 50 may continue to be pulled proximally while the pusher 4 is used to push forward or hold the body 10 in place. As the sheath 50 is withdrawn and more of the body 10 is freed from the sheath 50 the spacers 20 will spring out to assume a pre-formed shape. Should the sheath removal process cause the body 10 to shift forward, so that, e.g., the end 10b extends into the lumen of the artery, the tether 5 may be used to pull the body 10 back into the desired location shown in FIG. 3. When the body 10 is in the proper place, the tether 5 is removed from the connector 40 and the catheter is withdrawn.

As shown in FIG. 3, the connector 40 remains and is attached to the body 10 proximal end 10a for retrieval. When the flow deflector is no longer needed one of the disclosed retrieval systems (e.g., a catheter with distal snare) may be passed through the vein and ensnared with a hook portion of the connector 40 (FIG. 8A). With the connector 40 ensnared the flow deflector is removed and pulled downstream for removal from the patient.

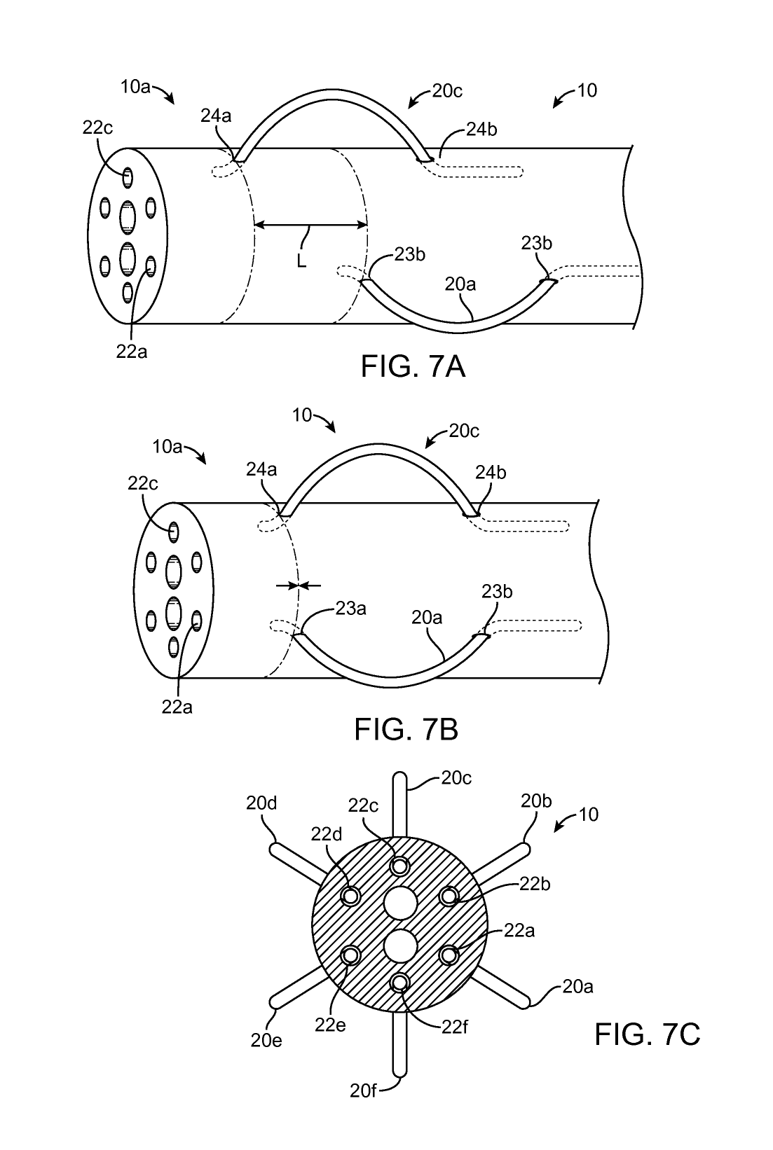

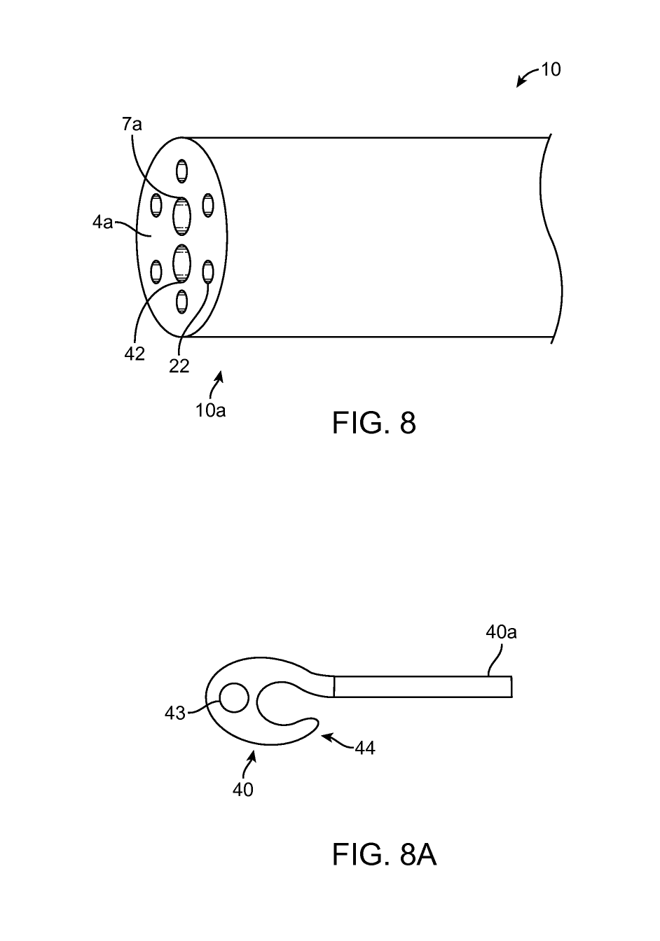

Referring to FIGS. 5A, 5B, 7C, 6A and 6B there are provided perspective and front views of the body distal end 10a and/or proximal end 10a and a further description of the stowed, deployed and lumens for the spacers 20 of the flow deflector according to preferred embodiments. FIG. 7C shows a cross-sectional and frontal view of the flow deflector, illustrating the body 10, which according to the illustrated embodiment has six lumens formed for receiving a shape memory wire material forming the spacers 20. The six lumens 22 are evenly spaced 60 degrees apart about the circumference of the body 10. Each lumen 22 form a passage or bore that may extend through the entire length of the body 10. Alternatively, the bores may extend only partially through as needed to accommodate the stowed spacer 20 (as more fully appreciated with reference to the description accompanying FIGS. 5A-5B and 6A-6B).

There are six spacers 20, each having its own bore 22 in FIG. 7C. Thus, this embodiment is similar to the embodiment of FIG. 3, in that there are six spacers 20a-20f used. However, in contrast to FIG. 3, each of the six spacers 20a-20f are angularly spaced from each other, whereas in FIG. 3 pairs of spacers 20 (i.e., each pair being a proximal end and distal end spacer) share the same angular position along the body 10 so that when viewed from the perspective of FIG. 7C only three spacers 20 (spaced 120 degrees apart) are viewable. For the embodiment shown in FIG. 3 there may therefore be three passages or bores 22 formed that are 120 degrees apart and extend through the length of the body 10.

As shown in FIG. 7C there are bores 22a, 22b, 22c, 22d, 22e and 22f provided in body 10 for each of the respective spacers 20a, 20b, 20c, 20d, 20e and 20f. The assembly of each of the spacers 20 within a bore 22 is described with reference to one of the spacers in a bore. The same description applies to all the spacers 20 shown in FIG. 7C.

Referring to FIGS. 5A-5B, there is shown an assembly of a spacer 20c within a bore 22c. Holes 24a, 24b are formed at the surface of the body 10 and extend into the open space of the bore 22c. The holes 24a, 24b are spaced apart according to the desired stiffness or the desired deployed height of the spacers 20. The wire extends out from the bore 22a at hole 24a then re-enters the bore 22c at the hole 24b. FIG. 5A shows the configuration of the spacer 20 when constrained within the sheath 50. FIG. 5B shows the configuration of the spacer 20 without the sheath 50 present. As can be appreciated from a comparison of FIGS. 5A with 5B, the spacer end 25b is free to slide along the bore 22c so that the spacer 20c is capable of assuming these two forms. The spacer 20c is fixed at end 25a and free to slide at the opposite end 25b. As such, the spacer 20c can extend outward to assume its pre-formed shape (FIG. 5B) when the sheath 50 constraint is withdrawn. And when the sheath 50 is slid over the body 10 the spacer 20c can be easily pushed into the bore 22c, thereby causing the end 25b to slide (from left to right in FIGS. 5A-5B). This cases the apex 27 formed in FIG. 5B to flatten out. The end 25a may be fixed using adhesive. Or end 25a may be held in place by wrapping or crimping the wire together with ends of other wire forming spacers 20 at the body end 10a and/or 10b.

The space between the holes 24a, 24b can be used as a guide to dictate the amount of stiffness of the spacer 20 for supporting the body 10 in the center of the lumen; thus, the closer the holes 24a, 24b the more stiff is the spacer and the farther apart the less stiff is the spacer. Alternatively, or additionally, the spacing of holes 24a, 24b may be selected to achieve the desired height of the spacer when deployed over a given length of the body 10. As will be appreciated, for a fixed body 10 length (or portion thereof) over which the spacer wire can extend in the stowed position, more or less maximum deployed spacer height (i.e., height “h” in FIGS. 6A-6B) is obtained by bringing the holes 24a, 24b closer or further apart from each other, respectively. Thus, it will be appreciated that for embodiments where there are six spacers (FIG. 3) the holes 24a, 24b would need to be closer to each other than in the case of the embodiment of FIG. 4, since there may be less space available for the wire to extend in the stowed configuration, thereby requiring shorter wires.

FIGS. 6A and 6B show two embodiments of wire forming spacers 20. The spacers 20c, 20c′ shown here take the shape as shown when the apex 27, 27′ abuts the inner walls of the blood vessel. The spacer 20c′ is formed from a longer piece of wire than spacer 20c. The longer wire spacer 20c′ may be preferred as it can be configured to extend outward further in response to growth of the vessel during maturation, yet not causing a chronic outward force problem. The height “h” in FIGS. 6A-6B depicts a distance from the surface 10c of the body to the vessel inner wall.

For a blood vessel inner diameter dv and body outer diameter db the height “h” is (dv−db)/2. For a vessel diameter (dv) that is 2, 3, and 4 times the body diameter (db) the height “h” may be at least to %*db, db and 2*db, respectively, when the wire is fully deployed within the lumen. Preferably the spacer 20 is made long enough so that the body 10 can be supported within the lumen prior to and after the vessel lumen has expanded in size, which may be up to 2-5 times the size of the vessel diameter before the fistula is made.

An average, or size of the body is about 2-3 mm. A vein can expand up to six times its normal size during maturation. TABLE 1, below, shows examples of the height “h” as defined above for vein sizes ranging from 2 to 6 mm, with respect to body sizes from 1-3 mm.

TABLE 1
vein inner diameter (mm)

| body 10 outer diameter (mm) | 2 | 3 | 4 | 5 | 6 |
|---|---|---|---|---|---|
| 1 | 0.5 | 1.0 | 1.5 | 2.0 | 2.5 |
| 2 | — | 5.5 | 1.0 | 1.5 | 2.0 |
| 3 | — | — | 0.5 | 1.0 | 1.5 |

It will be appreciated that a spacer should have an initial deployed size that extends further as the vein enlarges, but without causing any adverse effects on maturation of the vein (e.g., the spacer should be spring-biased outward but not with a spring force too high to cause damage to the vessel walls, such as when a super-elastic metal is used for spacers 20 and a chronic outward force problem develops. Also, the spacers should be arranged to provide a generally uniform radially outward force so as to discourage or avoid a non-circular growth of the vessel in response to uneven or non-circular radial forces imposed by the spacers).

In one example for a body of size 1 mm, when the flow deflector is initially placed there is a spacer height of about 0.5 to 1.0 mm. As the vein increases in size the spacer extends out further thereby increasing h. Thus, if the vein increases in size from 3 mm to 6 mm for a 1 mm body the height increases in size by 250% to compensate for a doubling of the vein inner diameter size.

FIGS. 7A-7B illustrates embodiments of longitudinal offsets among spacers 20. Shown are only spacers 20a, 20c from FIG. 7C (same description applies for other spacers). In FIG. 7A the spacers 20a, 20c are longitudinally spaced by a length L. In FIG. 7B the two spacers 70a, 70c are longitudinally aligned. An example of the offset shown in FIG. 7A is found in FIG. 2 and an example of the aligned spacers of FIG. 7B is found in FIG. 3 or 4. The alignment is with regard to the holes 24a, 23a and holes 24b, 23b. It will be appreciated that for different lumen sizes, lengths of a body 10 and aligned or longitudinally offset spacers 20 may be selected as needed.

It is preferred to have two or more spacers longitudinally offset from each other (e.g., helical) when the vessel into which the flow deflector is placed is tortuous. For straight vessels it is preferred to have spacers that are aligned with each other. The later vessel type is expected to have less flow dynamics tending to cause the body to more towards walls of the vessel, and/or the lack of a curved lumen requires less support over its length to maintain the body away from vessel walls.

Referring to Table 2, below, In view of the foregoing, there are the following four (4) embodiments of a flow deflector, with respect to arrangements of spacers 20 for spacing the body 10 away from the blood vessel walls:

TABLE 2

| | Number of | Angle between | Longitudinal spacing |
|---|---|---|---|
| | spacers | spacers | (with respect to distance between |
| | used | (FIG. 7C) | holes 24a, 24b - FIGS. 7A-7B) |
| 1 | 3 | 120 | Offset longitudinally over length (e.g., |
| | | | helical/FIG. 2) |
| 2 | 3 | 120 | Aligned longitudinal at proximal or |
| | | | distal end (FIG. 4) |
| 3 | 6 | 120 | Aligned longitudinal at both proximal |
| | | | and distal ends (FIG. 3) |
| 4 | 6 | 60 | Offset longitudinally over length (e.g., |
| | | | helical/FIG. 2) |

Referring to FIGS. 8, 8A there is shown the proximal end of the body 10 and the connector 40, respectively. At the body 10 proximal end 10a there is a surface 4a that makes contact with the pusher 4 surface 4b (FIG. 2) for pushing the body 10 distally of the catheter 2 during deployment/placement. Also shown is a lumen 7a for the guide wire 7 and hole 42 for receiving a butt end 40a of the connector 40.

The holes or lumen for the guide wire 7 and connector 40 may be located so that they are circumscribed by the lumen for the spacers 20. The lumen sizes for the guide wire 7 and connector 40 may be larger than the lumen formed for the spacers 20. The lumen may be formed when the body 10 is formed, e.g., when the body 10 is molded, or the lumen may be formed in a solid body 10 using a laser cutter. Or the body 10 may be formed by a collection of tubes that are joined together. According to one embodiment the body 10 is made from an extruded polymer tube.

Referring to FIG. 8A the connector 40 includes the butt end 40a that may be pressed into the hole 42 to secure the connector 40 in place at the proximal end 10a. The connector 40 forms an eyelet 43 for receiving the tether 5 of the catheter 2 and a hook 44 for engagement with a retrieval snare. When the flow deflector is delivered, the tether 5 passes through the eyelet and can be pulled taught to hold the body surface 4a against surface 4b of the pusher 4 during delivery. When the flow deflector is in proper position (FIG. 3) the catheter is separated therefrom by removing the tether 5 from the eyelet 43. In an alternative embodiment the connector 40 may be a looped wire disposed at the proximal end 10a.

This placement by the catheter 2, and separation of the flow deflector from the catheter 2 may be accomplished from a proximal end handle of the catheter 2 by a tether 5 so arranged that both ends of the tether 5 are disposed at the catheter 2 proximal end; thereby making both ends of the tether 5 accessible to the operator. When the tether 5 is pulled taught during delivery, both ends are secured together to hold the end 10a against the pusher 4. When the flow deflector is in place and the catheter 2 is to be separated from the flow deflector, the tether 5 is removed from the eyelet 43 by pulling proximally on only one end of the tether 5 so that the opposite end travels distally towards the eyelet 43 and eventually passes through the eyelet 43, thereby separating the tether 5 (and catheter 2) from the eyelet 43.

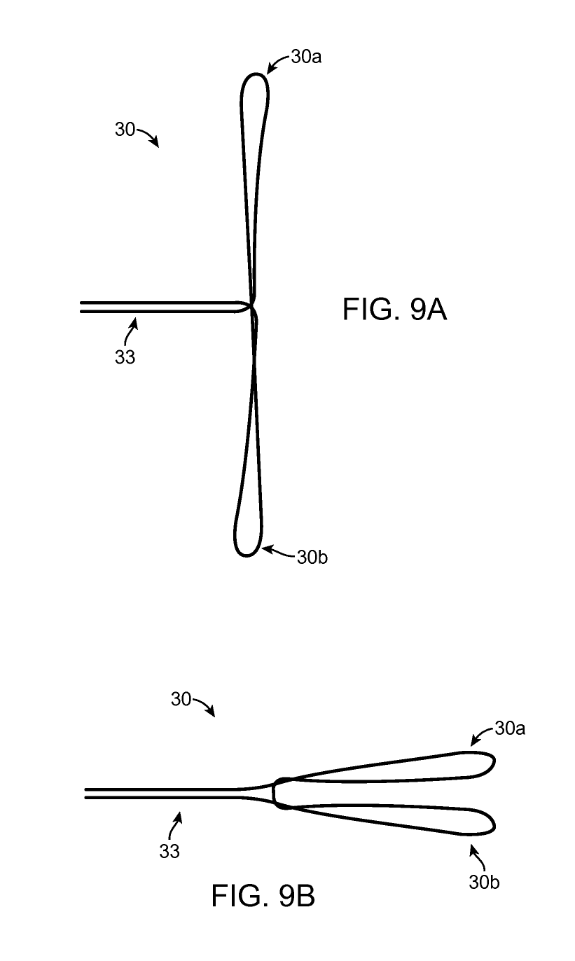

Referring to FIG. 9 there is shown the distal end 10b of the body 10 with the anchors 30a, 30b configured in their deployed position. The wire forming the anchors 30a, 30b is held within a hole 32 of the body 10. The wire may be secured by adhesive within the hole 32. For a polymer body 10 the anchors 30 may be molded-in with the body 10. Rather than have separately formed holes 32 and 42 for the anchor 30 and connector 40, respectively, a single lumen may be formed and extend from the distal to proximal end, as indicated in FIG. 9. This way the same lumen can be used to hold the anchor 30 at the distal end 10b and the connector 40 at the proximal end 10a. Also shown in FIG. 9 is a radiopaque marker 60 located at the distal end 10b. The marker is visible using fluoroscopy to locate the position of the distal end 10b of the body 10 for placement at the fistula.

Referring to FIGS. 9A-9B there is shown the deployed configuration (FIG. 9A) and stowed configuration (FIG. 9B) of the anchor 30. According to this embodiment the petals or wings 30a, 30b are made from a single piece of shape-memory material. The wire is pre-shaped as shown in FIG. 9A. Thus, with no external forces applied the wire will naturally take the shape shown in FIG. 9A. When confined within the sheath 50 the wire takes the shape shown in FIG. 9B. The ends 33 of the wire are secured within the hole 32 at the body 10 distal end 10b.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the claims should not be construed to limit the invention to the specific embodiments disclosed in the specification. Rather, the scope of the invention is to be determined entirely by the claims, which are to be construed in accordance with established doctrines of claim interpretation.

What is claimed is:

1. A flow deflector, comprising:
an elongate body having a distal end and a proximal end;
an anchor configured to extend distally of the distal end and radially outward from the body; and
a plurality of spacers configured to extend from a surface of the body.

2. The flow deflector of claim 1, wherein the body is made from a polymer, metal or metal alloy.

3. The flow deflector of claim 1, wherein the body includes a lumen for receiving a guide wire.

4. The flow deflector of claim 1, wherein the body is cylindrical.

5. The flow deflector of claim 4, wherein the body has a length to diameter ratio of about 20:1, 30:1, 40:1 or between about 20:1 to 30:1 or between about 20:1 to 40:1.

6. The flow deflector of claim 4, wherein the anchors are configured between a deployed and stowed form, wherein when in the stowed form the anchors have a maximum radial extent less than or equal to the diameter of the body.

7. The flow deflector of claim 1, wherein the anchors are made from a shape memory material.

8. The flow deflector of claim 1, wherein the anchors comprise a pair of wings or petals.

9. The flow deflector of claim 1, wherein the spacers are made from a shape memory material.

10. The flow deflector of claim 1, wherein at least three spacers are configured to extend from the body surface.

11. The flow deflector of claim 1, wherein a spacer includes a first end, second end and a center portion configured to extend from the body surface, wherein the first and second ends are disposed within the body and the center portion is exterior of the body surface.

12. The flow deflector of claim 11, wherein the first end is fixed to the body and the second end is disconnected from the body.

13. The flow deflector of claim 1, wherein a spacer is a wire that has first and second ends received in a lumen of the body, and a center portion configured to extend outward from the body surface.

14. The flow deflector of claim 13, wherein the center portion of the wire extends out from a first hole and into a second hole, the first and second holes each being formed in the body surface and in communication with the body lumen.

15. The flow deflector of claim 13, wherein the body has a length (L) and the first and second holes are separated by a length equal to about ½*L, ⅓*L, ¼*L, or between about ⅕*L and ½*L.

16. The flow deflector of claim 1, wherein the body has a diameter D and each one of, or any combination of the spacers are configured for radially extending from the body surface by up to about ½*D, 2*D, 3*D, 4*D, and *5D.

17. The flow deflector of claim 1, wherein the flow deflector has three or six spacers.

18. The flow deflector of claim 1, wherein a spacer is longitudinally and/or radially aligned with at least one other spacer or offset and/or radially spaced from at least one other spacer.

19. A flow deflector delivery system, comprising:
a flow deflector, including
an elongate body having a distal end and proximal end, and
a connector disposed at the proximal end; and
a catheter having a distal end, including
a tether engaged with the connector, and
a sheath encapsulating the flow deflector.

20. The system of claim 19, wherein the connector comprises a loop, a hook and/or an eyelet.
